(19) United States
(12) Patent Application Publication
(10) Pub. No.: US 2024/0415858 A1
(43) Pub. Date: Dec. 19, 2024

(54) PROMOTING MYELINATION BY N-ACETYLGLUCOSAMINE AND MODULATION OF N-GLYCAN BRANCHING

(71) Applicant: THE REGENTS OF THE UNIVERSITY OF CALIFORNIA, Oakland, CA (US)

(72) Inventors: Michael Demetriou, Irvine, CA (US); Michael Sy, Irvine, CA (US); Alexander Brandt, Irvine, CA (US)

(21) Appl. No.: 18/694,720

(22) PCT Filed: Sep. 23, 2022
(86) PCT No.: PCT/US2022/076953

§ 371 Date: Mar. 22, 2024

Related U.S. Application Data
(60) Provisional application No. 63/247,708, filed on Sep. 23, 2021.

(51) Int. Cl. A61K 31/7008; A61K 9/00; A61P 25/00

(52) U.S. Cl. CPC A61K 31/7008; A61K 9/0053; A61P 25/00

(57) ABSTRACT

The present invention features compositions and methods for the repair of myelin and/or treatment of demyelinating diseases. The demyelinating disease may be multiple sclerosis (MS), optic neuritis, myelin oligodendrocyte glycoprotein antibody associated disease, neuromyelitis optica spectrum disorder, transverse myelitis, or acute disseminated encephalomyelitis. The compositions may include N-acetylglucosamine (GIcNAc), GlcNAc-6-acetate, or a combination thereof. The method may involve administering the compositions to a subject in need.

### CROSS-REFERENCES TO RELATED APPLICATIONS

This application claims benefit of U.S. Provisional Application No. 63/247,708 filed Sep. 23, 2021, the specification of which is incorporated herein in its entirety by reference.

### STATEMENT REGARDING FEDERALLY SPONSORED RESEARCH OR DEVELOPMENT

This invention was made in part with government support under Grant No. R01AT007452 awarded by the National Center for Complementary and Integrative Health, Grant No. R01A1144403 awarded by the National Institute of Allergy and Infectious Disease. The government has certain rights in the invention.

### FIELD OF THE INVENTION

The present invention features compositions and methods for enhancing primary myelination, treatment of demyelination as well as compositions and methods for remyelination.

### BACKGROUND OF THE INVENTION

Myelination of axons by oligodendrocytes in the central nervous system plays a critical role in normal cognitive development and function as well as in demyelinating disease such as multiple sclerosis (MS). In addition to speeding conduction of the action potential, myelination supports axon health and survival. In MS, re-myelination of demyelinated axons by oligodendrocytes is often incomplete despite the presence of abundant oligodendrocyte precursor cells (OPC) throughout the brain. The molecular mechanisms that block re-myelination in MS are incompletely understood and there is a lack of therapies to promote myelin repair. Failure to adequately re-myelinate is influenced by the microenvironment of the MS lesion, where reactive astrocytes, microglia, and macrophages produce various inhibitory factors leading to disruption in OPC differentiation, oligodendrocyte migration, process outgrowth, and attachment to axons. Thus, increasing OPC differentiation has become one important strategy for promoting remyelination in MS and other demyelinating diseases.

Cell surface and secreted proteins are co- and post-translationally modified on Asn(N) by the addition of carbohydrates (N-glycans) in the endoplasmic reticulum and subsequently remodeled in the Golgi. The degree of N-acetylglucosamine (GIcNAc) branching in N-glycans promotes binding to galectins, a family of sugar-binding proteins (FIG. 5A). Poly-valent galectin—glycoprotein interactions at the cell surface form a macromolecular lattice that simultaneously controls the movement, clustering and/or endocytosis of multiple receptors and transporters to control signaling, cell growth, differentiation and death. For example, N-glycan branching controls epithelial cell growth by regulating receptor tyrosine kinases endocytosis, promotes glucose uptake in mesenchymal and pancreatic cells by inhibiting glucose transporter endocytosis and reduces T cells, B cell and neutrophil pro-inflammatory responses by co-regulating the clustering and/or endocytosis of multiple glycoproteins. These mechanisms in turn impact cancer, type II diabetes and autoimmunity. For example, reductions in N-glycan branching are associated with MS and promote both inflammatory demyelination and neurodegeneration in mice, the latter by an unknown mechanism. Therefore, given the diverse and pleiotropic effects of N-glycan branching, identifying and manipulating regulatory mechanisms may provide new insights into disease pathogenesis and opportunities for therapeutic intervention.

### BRIEF SUMMARY OF THE INVENTION

It is an objective of the present invention to provide compositions and methods that allow for enhancing primary myelination of the brain as well as the treatment and repair of myelin, as specified in the independent claims. Embodiments of the invention are given in the dependent claims. Embodiments of the present invention can be freely combined with each other if they are not mutually exclusive.

Myelination plays an important role in cognitive development during childhood and in demyelinating diseases like multiple sclerosis (MS), where failure of re-myelination promotes permanent neuro-axonal damage. Modification of cell surface receptors with branched N-glycans coordinates cell growth and differentiation by controlling glycoprotein clustering, signaling and endocytosis.

N-acetylglucosamine (GIcNAc) is a rate-limiting metabolite for N-glycan branching. As described herein the present invention demonstrates that GIcNAc and N-glycan branching trigger oligodendrogenesis from precursor cells by inhibiting PDGF receptor-α cell endocytosis. Supplying oral GIcNAc to lactating mice drives primary myelination in newborn pups via secretion in breast milk, while genetically blocking N-glycan branching markedly inhibits primary myelination. In adult mice with toxin (cuprizone) induced demyelination, oral GIcNAc prevents neuro-axonal damage by driving myelin repair. In MS patients, endogenous serum GIcNAc levels are inversely correlated with imaging measures of demyelination and microstructural damage. Additionally, the present invention identifies N-glycan branching and GIcNAc as critical regulators of primary myelination and myelin repair and suggests oral GIcNAc may be neuro-protective in demyelinating diseases like MS.

In some embodiments, the present invention features a method of treating a demyelinating disease in a subject in need of such treatment. In some embodiments, the method comprises administering to the subject a therapeutic amount of N-acetylglucosamine (GIcNAc). In certain embodiments, the method comprises intravenously administering to the subject a therapeutic amount of N-acetylglucosamine (GIcNAc).

The present invention may also feature a method of treating a demyelinating disease in a subject in need of such treatment. In some embodiments, the method comprises administering to the subject a therapeutic amount of a composition that promotes N-glycan branching. In some embodiments, the composition comprises N-acetylglucosamine (GIcNAc), GIcNAc-6-acetate, or a combination thereof. In some embodiments, the composition promotes N-glycan branches in neural stem cells, oligodendrocyte precursor cells, oligodendrocytes, or a combination thereof.

One of the unique and inventive technical features of the present invention is the use of GIcNAc for primary myelination and the repair of myelin in the brain. Without wishing to limit the invention to any theory or mechanism, it is believed that the technical feature of the present invention advantageously provides for the ability to enhance cognitive development and function through enhanced primary myelination as well as treating demyelinating diseases such as multiple sclerosis via remyelination. None of the presently known prior references or work has the unique inventive technical feature of the present invention.

Furthermore, the prior references teach away from the present invention. For example, GIcNAc has previously been shown to inhibit T cell function and prevent T cell mediated demyelination (PMID: 17488719, PMID: 21965673, PMID: 21629267, PMID: 28059703). The ability to impact neural stem cells/oligodendrocyte precursor cells and myelination/remyelination is not taught or obvious based on this prior knowledge.

Furthermore, the inventive technical feature of the present invention contributed to a surprising result. For example, the current invention shows for the first time that GIcNAc can cross the blood brain barrier to trigger neural stem cell/oligodendrocyte precursor cell differentiation to oligodendrocytes and subsequent myelination. Furthermore, the current invention shows for the first time that GIcNAc secreted in breast milk can be taken up by the nursing infant, cross the blood brain barrier of the infant and promote primary myelination in the developing brain. These activities could not have been foreseen by its known activities on T cells. Further, the current invention found oral delivery of GIcNAc to humans is limited by the bowel microbiota metabolizing GIcNAc, as evidenced by excess gas, bloating and loose stool in 50% of subjects at the 12 g dose (i.e., 4 g three time per day) but not at 6 g dose (ie 2 g three times per day). This teaches that to further enhance GIcNAc levels in humans, systemic delivery (e.g., intravenous) is required to bypass the effects of the gut microbiota.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, 1D, and 1E show GIcNAc and N-glycan branching promotes oligodendrogenesis. FIGS. 1A, 1B, 1C and 1D show flow cytometry of E12.5 NSC's (neural stem cells) from CD1 (FIGS. 1B and 1C) or C57BL/6 (FIG. 1D) mice cultured in growth media (FGF+EGF)±GIcNAc for 48 hours. Cell surface binding levels of L-PHA and PDGFRα are measured as mean fluorescence intensity (MFI). FIG. 1E shows flow cytometry and immunofluorescence microscopy of E12.5 NSC's in differentiation media (FGF+PDGF−AA) from Mgat5^{+/+}, Mgat5^{+/-} and Mgat5^{-/-} C57BL/6 mice. Data are 3 technical replicates per group (FIG. 1A-1E), representative of 3 (FIG. 1A-1C) or 2 (FIGS. 1D and 1E) experiments. P-values are by one-way ANOVA with Sidaks's multiple comparison test. All error bars are standard error. *p<0.05, **p<0.01, ***p<0.001, ****p<0.0001.

FIGS. 2A, 2B, 2C, 2D, and 2E show GIcNAc and N-glycan branching promote primary myelination. FIGS. 2A, 2B, and 2C show newborn PL/J mice were given exogenous GIcNAc or [U¹³C]GIcNAc by providing their nursing mothers with GIcNAc or [U¹³C]GIcNAc at 1 mg/mL in drinking water from P3-P8. The pups and mothers were sacrificed at P8 and brains were analyzed by LC-MS/MS for UDP-[U¹³C]HexNAc (FIG. 2A, N=3,3 adult, N=4,4 P8 pups), flow cytometry (FIG. 2B, N=3,3) or immunofluorescence microscopy (FIG. 2C, N=5,5) for the indicated markers. L-PHA staining in FIG. 2B is gated on PDGFRα⁺ cells. Data in FIG. 2C is the average fluorescence intensity of the area depicted in red of 3 brain slices per mouse. One-sided t-test. FIGS. 2D and 2E shows the indicated adult mice (10 weeks old) were treated with tamoxifen at week 0 and 4, sacrificed at week 8 and brains were analyzed by immunofluorescence microscopy for MBP, myelin (fluoromyelin), Olig2⁺ and CC1⁺ cells (N=5 (2 male, 3 female), 8 (6 male, 2 female) (FIG. 2D) and N=5 (2 male, 3 female), 4 (2 male, 2 female) (FIG. 2E); one-sided t-test). Each data point in the graphs represents average fluorescence or cell counts of the highlighted area from 3 (FIG. 2D) or 2 (FIG. 2E) different brain slices per mouse.

FIGS. 3A, 3B, 3C, 3D, 3E, 3F, and 3G show Oral GIcNAc promotes re-myelination and limits axonal injury. FIG. 3A shows the ability of GIcNAc to promote myelin repair in vivo was assessed using the cuprizone model, with oral GIcNAc treatment (1 mg/mL in drinking water) during the last 3 weeks of a 6-week cuprizone exposure (I, Active phase treatment) or after 5 weeks of cuprizone treatment for 1, 2 or 4 weeks (II, III, IV, Recovery phase treatment). WT (I, III) or Mgat5 heterozygous (II, IV) C57BL/6 mice were used. FIG. 3B shows the area of the medial corpus callosum (CC) analyzed. FIGS. 3C, 3D, 3E, and 3F shows the latency to fall in an accelerated rotarod test as well as immunofluorescence staining of the CC for MBP, degraded MBP (dMBP), APP, myelin (fluoromyelin) and/or CC1/Olig2 from wildtype mice with active phase GIcNAc treatment (FIG. 3C, N=5,5, all male), Mgat5 heterozygous mice with recovery phase GIcNAc treatment for 1 week (FIG. 3D, N=8 (5 male, 3 female), 7 (4 male, 3 female)), wildtype mice with recovery phase GIcNAc treatment for 2 weeks (FIG. 3E, N=11,14 for rotarod, N=5,7 for immunofluorescence, all male), or Mgat5 heterozygous mice with active phase GIcNAc for 4 weeks (FIG. 3F, N=6,6 with 4 male and 2 females per group). Data points represents average fluorescence from 3-4 different brain slices per mouse. Rotarod p-values by 2-way ANOVA with Sidak's multiple comparisons post test. Immunofluorescence p-values by one-tailed t-test. Scale bar=50 μm. FIG. 3G shows the CC of mice from the 4 week recovery phase treatment group in FIG. 3F were analyzed by electron microscopy (N=3,3 with 2 male and 1 female per group). Representative electron micrographs in control and GIcNAc treatment groups are shown, scale bar=1 μm. Filled and empty arrowheads indicate examples of myelinated and unmyelinated dystrophic axons, respectively. Plot of g-ratio vs. axon diameter (N=214 and 222 axons) was counted blindly from 2 fields (105 μm²) per mouse (p-value comparing best fit curves from non-linear regression, R² is the goodness of fit for each group). Numbers of total axons, myelinated axons, and dystrophic axons (axon diameter>0.7 μm) were counted blindly in 6 fields (105 μm²) per mouse in each treatment group (N=18, 18, p-value by one-sided t-test). All error bars are standard error.

FIGS. 4A, 4B, and 4C show Serum HexNAc correlates with markers of myelin-axon microstructural damage in MS patients. FIGS. 4A, 4B, and 4C show the association of serum HexNAc levels with Magnetic Resonance Imaging (MRI) measures of myelin-axon microstructural damage in a cohort of n=180 MS patients. T2w lesion volume (FIG. 4A) and T1w/T2w ratio in Normal Appearing White Matter (NAWM, FIG. 4B) and Grey Matter (GM, FIG. 4C) is shown. Coefficient B, Standard Error (SE), and R² are from linear regression models correcting for age and sex (in FIGS. 4B and 4C). Black lines in regression models represent coefficients from non-corrected models, grey areas show the 95% confidence interval. Values in nM are serum HexNAc levels.

FIGS. 5A, 5B, 5C, 5D, 5E, 5F, and 5G show GIcNAc promotes oligodendrogenesis from precursor cells. FIG. 5A shows UDP-GIcNAc, which is synthesized de novo from glucose or salvaged from GIcNAc, is the donor substrate utilized by the Mgat branching enzymes. FIG. 5B shows immunofluorescence microscopy of E12.5 NSC's from CD1 mice cultured in growth media (FGF+EGF)±GIcNAc for 48 hours.

FIGS. 5C, 5D, 5E, 5F, and 5G shows flow cytometry of C57BL/6 mouse E12.5 NSC's of the indicated genotypes treated in either growth media (FGF+EGF, FIG. 5C), in differentiation media (FGF+PDGF−AA (10 ng/mL)) (FIG. 5E-5G) or as indicated (FIG. 5D) with/without GIcNAc 80 mM (FIG. 5C-5E), kifunensine for 48 hrs (FIG. 5F) or doxycycline pre-treatment for 8 days (FIG. 5G). Data are 3 technical replicates per group and representative of 2 experiments. P-values are by one-way ANOVA with Sidaks's multiple comparison test. (*p<0.05, **p<0.01, ***p<0.001, ****p<0.0001).

FIGS. 6A, 6B, 6C, 6D, and 6E show GIcNAc and N-glycan branching promote primary myelination. FIG. 6A shows flow cytometry of brains from PL/J pups whose mothers were treated with GIcNAc (1 mg/mL) in drinking water from P5 or E12.5 to P8 (N=5,5 and 2, 11; one sided t-test). FIG. 6B shows Mgat1^{f/f}Plp1-cre/ERTc⁺ mice were injected with or without tamoxifen (75 mg/kg) daily for 3 days and brains analyzed by flow cytometry. Data is representative of 3 mice per group. FIG. 6C shows Mgat1^{f/f}Plp1-cre/ERTc⁺ mice (10 weeks old) were treated with tamoxifen at week 0 and 4, sacrificed at week 8 and cerebellums were analyzed by immunofluorescence microscopy for MBP and myelin (fluoromyelin) (N=5 (2 male, 3 female),8 (6 male, 2 female); one sided t-test). Data points represent average fluorescence of area depicted from 2 slices per mouse. FIGS. 6D and 6E show that the indicated mice (10 weeks old) were treated with tamoxifen at week 0 and then sacrificed at week 2 and brains were analyzed by immunofluorescence microscopy for MBP and myelin (fluoromyelin) (N=8 (4 male, 4 female), 6 (4 male, 2 female (FIG. 6D), N=8 (4 male, 4 female), 4 (1 male, 3 female) (FIG. 6E), one sided t-test. Data points represent average fluorescence of areas highlighted in red in 2 slices per mouse.

FIGS. 7A, 7B, and 7C show Oral GIcNAc promotes oligodendrogenesis and re-myelination while limiting axonal injury. FIG. 7A shows per mouse averages of g-ratio, total axons, myelinated axons, paranodes, and dystrophic axons as well number of degenerating axons from electron micrographs of the medial CC of the Mgat5—mice from FIG. 3G (N=3,3 (2 male and 1 female per group)) with data obtained blind to treatment conditions and averaged from 2 fields (g-ratio) or 6 fields per mouse (105 μm/field). Degenerating axons highlighted by arrows and paranodes denoted by asterisks. Mice were treated ±GIcNAc for 4 weeks after 5 weeks of cuprizone. P-value by one-sided t-test and chi-square test. Scale bar=1 μm. FIG. 7B shows electron micrographs of 10-week old Mgat5^{+/+} and Mgat5^{+/-} littermates were analyzed for number of myelinated axons per field (N=12, 12 fields; 6 fields per mouse, 105 μm/field, one-sided t-test) and g-ratio (N=839 axons over 6 fields). Scale bar=1 μm. FIG. 7C shows fluoromyelin staining of medial CC of Mgat5^{+/-} mice who were treated ±GIcNAc for 6 weeks after 5 weeks of cuprizone starting at 10 weeks of age (N=3 (3 male), 6 (4 male, 2 female); one sided t-test). Data points represent an average of 4 slices per mouse.

FIGS. 8A, 8B, 8C, 8D, 8E, 8F, 8G, 8H, 8I, and 8J show Oral GIcNAc raises serum HexNAx and N-glycan branching in activated T-cells of MS patients. FIG. 8A shows baseline blood was drawn weekly four times prior to treatment, followed immediately by four weekly blood draws after oral GIcNAc treatment at 3 g, 6 g, or 12 g per day (divided into three doses). For the 6 g and 12 g cohorts this was followed by a washout period and three additional blood draws. FIGS. 8B, 8C, 8D, 8E, 8F, and 8G shows serum HexNAc levels were measured using LC-MS/MS and pre-GIcNAc, GIcNAc +/−post GIcNAc periods compared by i) averaging the weekly HexNAc measurements from single patients before, during +/−after treatment with single patient response connected by black lines (FIG. 8B-8D) or ii) averaging all subjects each week with group response depicted (FIG. 8E-8G). P-values by 2-tailed Wilcoxon matched-pairs signed rank test. FIG. 8H-8J shows L-PHA flow cytometry of CD23+CD4+ T-cells blasts (based on gating larger cells on FSC vs SSC) of the average weekly MFI from single patients before, during +/−after treatment (the a priori method is outlined in the trial protocol). Single patient responses are connected by black lines. Data is normalized to pre-treatment and shown as percent change. P-values by 1-tailed Wilcoxon matched-pairs signed rank test.

FIG. 9 shows Oral GIcNAc reduces serum Nfl levels in MS patients with elevated baseline. Comparison of the change in serum Nfl concentration in subjects with elevated baseline levels (combined from the 3 g, 6 g, and 12 g cohorts) showing change from pre-GIcNAc to during weeks 3-4 of GIcNAc treatment. The four data points pre-GIcNAc (black dots) and the two data points from week 3 and 4 during GIcNAc treatment are averaged, normalized to pre-GIcNAc levels and shown as black and red dots connected by black lines for each subject, respectively. Nfl was measured by an ultra sensitive single molecule array immunoassay. P-value by 1-tailed Wilcoxon matched-pairs signed rank test.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult and newborn subjects, as well as fetuses, whether male or female, are intended to be included. In one embodiment, the subject is a mammal (e.g., a human) having a disease, disorder or condition described herein. In another embodiment, the subject is a mammal (e.g., a human) at risk of developing a disease, disorder or condition described herein. A “patient” is a subject afflicted with a disease or disorder. The term “patient” includes human and veterinary subjects. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., GIcNAc) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., demyelinating diseases), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of GIcNAC described herein is an amount sufficient to provide a therapeutic benefit in the treatment or management of a demyelinating disease, or to delay or minimize one or more symptoms associated with demyelinating diseases. A “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A therapeutically effective amount of GIcNAc described herein means an amount of therapeutic agent, alone or in combination with other therapies, which provides a therapeutic benefit in the treatment or management of demyelinating disease.

The terms “administering”, and “administration” refer to methods of providing a pharmaceutical preparation to a subject. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein.

The pharmaceutical formulation can be administered in a number of ways depending on whether local or systemic treatment is desired, and on the area to be treated. A preferred mode of administration of the composition is parenterally. Other modes of administration may be orally, topically, or parenterally, for example by intravenous drip, subcutaneous, intraperitoneal or intramuscular injection. The disclosed compounds can be administered intravenously, intraperitoneally, intramuscularly, subcutaneously, intracavity, transdermally.

As used herein a “demyelinating disease” is any condition that results in damage to the protective covering (myelin sheath) that surrounds nerve fibers in your brain, optic nerves and spinal cord.

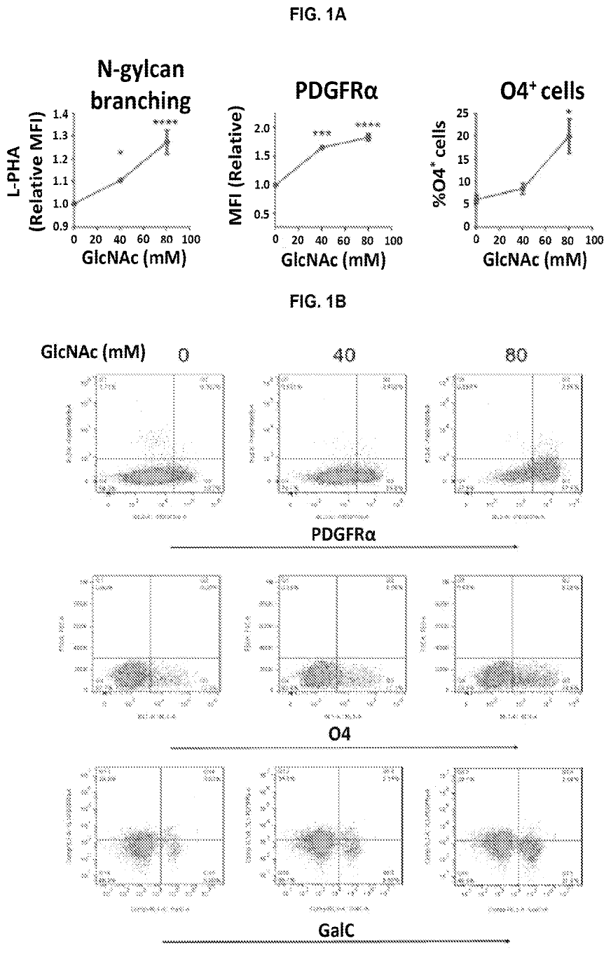

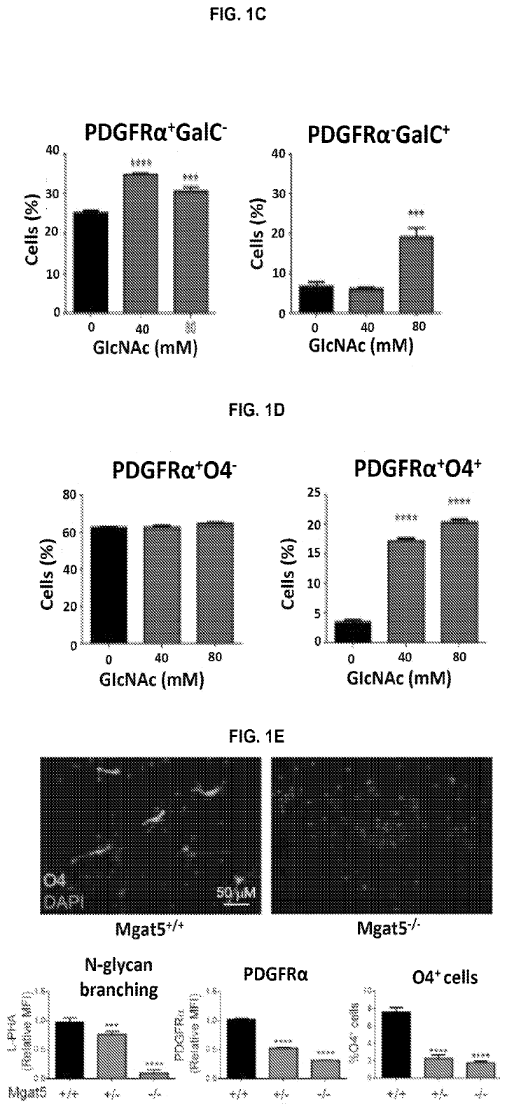

Referring now to FIGS. 1A-9, the present invention features compositions and methods for the prevention and treatment of demyelination as well as the restoration of myelin.

The present invention features a method of treating a demyelinating disease in a subject in need of such treatment. In some embodiments, the method comprises administering to the subject a therapeutic amount of N-acetylglucosamine (GIcNAc). In certain embodiments, the method comprises intravenously administering to the subject a therapeutic amount of N-acetylglucosamine (GIcNAc).

In some embodiments, non-limiting examples of a demyelinating disease includes but is not limited to multiple sclerosis (MS), optic neuritis, myelin oligodendrocyte glycoprotein antibody associated disease, neuromyelitis optica spectrum disorders, transverse myelitis, or acute disseminated encephalomyelitis.

In preferred embodiments GIcNAc is administered intravenously. In some embodiments, GIcNAc is administered intravenously at a dosage of about 1 g to 150 g, or about 1 g to 125 g, or about 1 g to 100 g, or about 1 g to 75 g, or about 1 g to 50 g, or about 1 g to 25 g, or about 25 g to 150 g, or about 25 g to 125 g, or about 25 g to 100 g, or about 25 g to 75 g, or about 25 g to 50 g, or about 50 g to 150 g, or about 50 g to 125 g, or about 50 g to 100 g, or about 50 g to 75 g, or about 75 g to 150 g, or about 75 g to 125 g, or about 75 g to 100 g, or about 100 g to 150 g, or about 100 g to 125 g, or about 125 g to 150 g. For example, GIcNAc may be administered at a dosage of about 1 g to 150 g with a preferred range of about 90 g to 110 g for administration intravenously.

Although intravenous administration is the preferred route of administration, a properly adjusted oral dosing may be effective in preventing or treating demyelinating diseases as described herein.

In other embodiments, GIcNAc is administered orally. In some embodiments, GIcNAc is administered orally at a dosage of about 1 g to 20 g, or about 1 g to 15 g, or about 1 g to 12 g, or about 1 g to 10 g, or about 1 g to 8 g, or about 1 g to 6 g, or about 1 g to 4 g, or about 1 g to 3 g, or about 1 g to 2 g, or about 2 g to 10 g, or about 2 g to 8 g, or about 2 g to 6 g, or about 2 g to 4 g, or about 2 g to 3 g, or about 3 g to 10 g, or about 3 g to 8 g, or about 3 g to 6 g, or about 3 g to 4 g, or about 4 g to 10 g, or about 4 g to 8 g, or about 4 g to 6 g, or about 6 g to 10 g, or about 6 g to 8 g, or about 8 g to 10 g. For example, the dosage may range from about 1 g to 15 g, with a preferred range of 3 g to 6 g for administration orally.

In some embodiments, GIcNAc may be administered once daily or twice daily. In other embodiments, GIcNAc may be administered three or four times daily. In further embodiments, GIcNAc may be administered once to four times daily; or GIcNAc may be administered at least once daily, at least once every other day, or at least once weekly. In some embodiments, GIcNAc may be administered continuously. In further embodiments, the composition may be administered orally or intravenously.

The present invention may also feature a method of treating a demyelinating disease in a subject in need of such treatment. In some embodiments, the method comprises intravenously administering to the subject a therapeutic amount of N-acetylglucosamine (GIcNAc).

The present invention also features a method of improving early brain development in an infant. In some embodiments, the method comprises orally administering to the infant a therapeutic amount of N-acetylglucosamine (GIcNAc).

In some embodiments, GIcNAc is incorporated into baby formula. In other embodiments, GIcNAc is used to supplement baby formula. Without wishing to limit the present invention to any theories or mechanisms it is believed that supplementing baby formula with GIcNAc will reduce the necessity of breast feeding, which currently improves myelination, cognition and brain development in infants relative to formula fed infants. In some embodiments, formula supplemented with GIcNAc improves myelination in the infant's brain. In other embodiments, formula supplemented with GIcNAc improves cognitive function in the infant. In further embodiments, formula supplemented with GIcNAc improves brain development in the infant.

The present invention may also feature a method of repairing myelin in a subject in need thereof. In some embodiments, the method comprises administering to the subject a therapeutic amount of N-acetylglucosamine (GIcNAc).

The present invention may further feature a composition comprising N-acetylglucosamine (GIcNAc) for use in a method for the treatment of a demyelinating disease. In another embodiment, the present invention features a composition comprising N-acetylglucosamine (GIcNAc), GIcNAc-6-acetate, or a combination thereof for use in a repairing myelin.

The present invention may also feature a method of treating a demyelinating disease in a subject in need of such treatment. In some embodiments, the method comprises administering to the subject a therapeutic amount of a composition that promotes N-glycan branching. In other embodiments, the method comprises administering to the subject a therapeutic amount of a composition that promotes N-glycan branching in neural stem cells, oligodendrocyte precursor cells, oligodendrocytes, or a combination thereof.

In some embodiments, the composition comprises N-acetylglucosamine (GIcNAc), GIcNAc-6-acetate, or a combination thereof. In some embodiments, the composition promotes N-glycan branches in neural stem cells, oligodendrocyte precursor cells, oligodendrocytes, or a combination thereof.

In preferred embodiments, compositions described herein (e.g., N-acetylglucosamine (GIcNAc) or GIcNAc-6-acetate) is administered intravenously. In some embodiments, compositions described herein is administered intravenously at a dosage of about 1 g to 150 g, or about 1 g to 125 g, or about 1 g to 100 g, or about 1 g to 75 g, or about 1 g to 50 g, or about 1 g to 25 g, or about 25 g to 150 g, or about 25 g to 125 g, or about 25 g to 100 g, or about 25 g to 75 g, or about 25 g to 50 g, or about 50 g to 150 g, or about 50 g to 125 g, or about 50 g to 100 g, or about 50 g to 75 g, or about 75 g to 150 g, or about 75 g to 125 g, or about 75 g to 100 g, or about 100 g to 150 g, or about 100 g to 125 g, or about 125 g to 150 g. For example, compositions described herein may be administered at a dosage of about 1 g to 150 g with a preferred range of about 90 g to 110 g for administration intravenously.

Although intravenous administration is the preferred route of administration, a properly adjusted oral dosing may be effective in promoting N-glycan branching in neural stem cells, oligodendrocyte precursor cells, oligodendrocytes, or a combination thereof.

In other embodiments, compositions described herein (e.g., N-acetylglucosamine (GIcNAc) or GIcNAc-6-acetate) is administered orally. In some embodiments, compositions described herein is administered orally at a dosage of about 1 g to 20 g, or about 1 g to 15 g, or about 1 g to 12 g, or about 1 g to 10 g, or about 1 g to 8 g, or about 1 g to 6 g, or about 1 g to 4 g, or about 1 g to 3 g, or about 1 g to 2 g, or about 2 g to 10 g, or about 2 g to 8 g, or about 2 g to 6 g, or about 2 g to 4 g, or about 2 g to 3 g, or about 3 g to 10 g, or about 3 g to 8 g, or about 3 g to 6 g, or about 3 g to 4 g, or about 4 g to 10 g, or about 4 g to 8 g, or about 4 g to 6 g, or about 6 g to 10 g, or about 6 g to 8 g, or about 8 g to 10 g. For example, the dosage may range from about 1 g to 15 g, with a preferred range of 3 g to 6 g for administration orally.

In some embodiments, compositions described herein (e.g., N-acetylglucosamine (GIcNAc) or GIcNAc-6-acetate) may be administered once daily or twice daily. In other embodiments, compositions described herein (e.g., N-acetylglucosamine (GIcNAc) or GIcNAc-6-acetate) may be administered three or four times daily. In further embodiments, compositions described herein (e.g., N-acetylglucosamine (GIcNAc) or GIcNAc-6-acetate) may be administered once to four times daily; or compositions described herein (e.g., N-acetylglucosamine (GIcNAc) or GIcNAc-6-acetate) may be administered at least once daily, at least once every other day, or at least once weekly. In some embodiments, compositions described herein (e.g., N-acetylglucosamine (GIcNAc) or GIcNAc-6-acetate) may be administered continuously. In further embodiments, the composition may be administered orally or intravenously.

### Example

The following is a non-limiting example of the present invention. It is to be understood that said example is not intended to limit the present invention in any way. Equivalents or substitutes are within the scope of the present invention.

### 1.1.1: Mouse Brain and Neural Stem Cell Isolation and Analysis:

Mice were bred and utilized as approved by the University of California, Irvine Institutional Animal Care and Use Committee. Dorsal forebrain cortical tissue was dissected from the medial ganglionic eminence (MGE) at embryonic day 12.5 (E12) of CD1 mice (Charles River) or Mgat5−/− C57BL/6 mice and their wildtype littermates and placed in dissection buffer comprising PBS, 0.6% glucose, and 50 U/mL Pen/Strep. Tissue from multiple embryos within the same litter were pooled, and a subsequent culture from a single litter was considered a biological repeat. The tissue was dissociated using 0.05% Trypsin-EDTA at 37° C. for 10 min, followed by treatment with soybean trypsin inhibitor (Life Technologies). Dissociated cells were re-suspended in proliferation medium comprising DMEM, 1× B27, 1× N2, 1 mM sodium pyruvate, 2 mM L-glutamine, 1 mM N-acetylcysteine, 20 ng/mL EGF (PeproTech), 10 ng/mL bFGF (PeproTech), and 2 μg/mL heparin and seeded at 150,000 cells/mL (non-tissue culture treated plastic plates) and grown as non-adherent spheres. Cell cultures were passaged approximately every 3 days using enzyme-free NeuroCult Chemical Dissociation Kit (Mouse) (StemCell Technologies). Cultures were passaged at least once prior to experimental use. For experiments, passaged cells were cultured in proliferation media (bFGF and EGF) or differentiation media (bFGF (10 ng/ml) and PDGF-AA (10 ng/ml); Life technologies) for 48 hours with or without the presence of GIcNAc (Ultimate Glucosamine, Wellesley Therapeutics) or kifunensine (GlycoSyn). Neurospheres were dispersed using the enzyme-free NeuroCult kit before being analyzed by flow cytometry using one or more of the following antibodies: anti-CD140a/PDGF-RA PE conjugate (1:200, A15785, Molecular Probes), anti-04 Alexa Fluor 488 conjugated (1:200, FAB1326G, RnD systems), Anti-GaIC Alexa Fluor 647 1:200, MAB342-AF647, Millipore), Anti-Olig2 (1:200, AB9610, Millipore) with anti-rabbit Alexa Fluor 488 (1:200, thermofisher).

GIcNAc treatment of mouse pups—GIcNAc (1 mg/mL) in drinking water was provided to pregnant PLJ mothers or mothers who recently delivered pups and were nursing their young. After the treatment period, pups were anesthetized with isoflurane and cardiac perfused with PBS. Pup and fetal brains were removed and homogenized by trituration using glass pipettes in PBS with 5% Fetal Bovine Serum (FBS).

Cells were then stained with antibodies and analyzed by flow cytometry using antibodies described herein. For immunofluorescence analysis of pup brains, pups were quickly decapitated, brains harvested and fixed in 4% paraformaldehyde overnight.

1.1.2: [U¹³C]GIcNAc treatment of mice:
[U¹³C] GIcNAc was purchased from Omicron Biochemicals and put in the drinking water at 1 mg/mL of female mice aged 8 weeks for 3 days. Fresh solution of [U¹³C] GIcNAc in drinking water was provided each day. After 3 days, mice were anesthetized with isoflurane and underwent cardiac perfusion with 50 mL of PBS. Brains were harvested and snap frozen in liquid nitrogen. Tissues were cut into 0.04 g pieces and crushed mechanically before undergoing extraction as described herein (see Example 1.1.10 Targeted LC-MS/MS). Levels of UDP-[U¹³C]GIcNAc were measured by LC-MS/MS analysis as described herein (Targeted LC-MS/MS).
1.1.3:Tamoxifen induced deletion of Mgat1:

Mgat1^{f/f}Plp1-cre/ERTc⁺ and Mgat1^{f/f}Pdgfra-creER⁺ were generated by crossing a Mgat1^{f/f}mice with Plp1-cre/ERTc⁺ and Pdgfra-creER⁺ lines from Jackson's Laboratory. Tamoxifen was dissolved in corn oil overnight at 37° C. at a concentration of 20 mg/mL. Mgat1^{f/f}Plp1-cre/ERTc⁺ and Mgat1^{f/f}Pdgfra-creER⁺ (mean age: P71.24, std. dev. 1.393) mice and their control Mgat1^{f/f} littermates were injected intraperitoneally with tamoxifen (75 mg/kg) daily for 3 days starting on day 0 and sacrificed at two weeks or re-treated with tamoxifen and sacrificed at 8 weeks. Mice were sacrificed following anesthesia and cardiac perfusion with phosphate buffered saline. Brains examined by flow cytometry were first homogenized by trituration using glass pipettes in PBS with 5% FBS. Brains examined for myelin content were drop fixed in 4% paraformaldehyde overnight.

### 1.1.4: Cuprizone Induced Demyelination:

Cuprizone at 0.2% induces demyelination in the corpus callosum by 3 weeks, with maximum demyelination at 5-6 weeks. 8-week-old C57BL/6 mice purchased from Jackson Laboratories or 8-week old Mgat5^{+/-} C57BL/6 mice were treated with 0.2% Cuprizone (Sigma) mixed into milled rodent chow for 6 weeks for the active phase treatment and 5 weeks for the recovery treatment. During active phase treatment, GIcNAc (1 mg/mL) in drinking water or just drinking water (control) was provided for the last 3 weeks of Cuprizone treatment. For the recovery phase treatment, GIcNAc in drinking water or control was provided after Cuprizone treatment had been stopped. Mice were anesthetized and underwent cardiac perfusion with 4% paraformaldehyde in PBS or 4% paraformaldehyde plus 0.5% glutaraldehyde in Sodium cacodylate buffer for immunofluorescence or electron microscopic analysis respectively. Brains were then fixed overnight in perfusion solution

### 1.1.5: Accelerated Rotarod:

One day prior to Cuprizone treatment, mice were trained on the rotarod by allowing them to run three 5-minute trials at a constant 30 rotations per minute (RPM). Mice then underwent weekly testing during Cuprizone and GIcNAc treatment on an accelerating rotarod starting at 4 rpm increasing to 40 rpm over 5 minutes. Latency for mice to fall was recorded. If a mouse was not running on the rotarod by holding on for 3 turns, this was considered a fall. For the active phase treatment, one trial was run every week. For the recovery phase treatment, 3 trials were run for each mouse each week and latencies were averaged. As expected with Cuprizone treatment performance degraded as treatment progressed. Mice whose performance did not drop below a predetermined threshold (200 seconds) were not used in analysis.

### 1.1.6: Immunofluorescence Analysis:

For neural stem cell (NSC) immunofluorescence, whole neurospheres were seeded onto laminin-coated coverslips (Neuvitro) in proliferation medium. After 24 hours, proliferation media was removed and replaced with differentiation medium (same components as proliferation medium but excluding EGF, bFGF, and heparin) to induce differentiation. For analysis of mouse brains, brains were incubated in 30% sucrose for at least 72 hours, embedded in OCT (Tissue-tek), frozen for at least 48 hours at −80° C., and then cut at 40 microns on a cryostat. Multiple sections from −1 bregma to −2.5 bregma were then stained with antibodies for MBP (1:100; MAB386, Millipore), Olig2 (1:200; AB9610, Millipore), CC1 (1:100; OP90, Millipore), degraded MBP (1:200, AB5864, Millipore) and Amyloid Precursor Protein (APP, 1:200; clone 22C11). After overnight incubation with primary antibody, tissues were washed and incubated with secondary antibodies: goat anti-rat Alexa-fluor 488 (1:200, Thermofisher), goat anti-rabbit TxRd (1:200, Thermofisher). APP is a marker for neuro-axonal damage while co-staining for CC1 and Olig2 are markers of mature oligodendrocytes. In order to examine the amount of myelin, slices were incubated in Fluoromyelin (1:300; F34651, Thermofisher) for 45 minutes. Images were acquired on a Keyence fluorescence microscope. Mean fluorescence intensity of the medial corpus callosum was measured using ImageJ.

### 1.1.7: Electron Microscopy (EM):

Three mice from each treatment group (control and GIcNAc) were selected randomly for EM analysis (before other investigations were performed). Portions of these brains from 0 to −1 bregma were rinsed in 0.1 M cacodylate buffer overnight and again for 15 min. the next day. 2×1 mm blocks of the corpus callosum were dissected out and post-fixed with 1% osmium tetroxide in 0.1 M cacodylate buffer for 1 hour, rinsed in ddH20, dehydrated in increasing serial dilutions of ethanol (70%, 85%, 95%, 100%×2) for 10 min each, put in propylene oxide (intermediate solvent) for 2×10 min, incubated in propylene oxide/Spurr's resin (1:1 mix) for 1 h, and then in Spurr's resin overnight. The blocks were put in a fresh change of resin in flat embedding molds the next day and polymerized overnight at 60° C. The blocks were sectioned at 1 μm using a Leica Ultracut UCT ultramicrotome. Floating sections were stained in toluidine blue (1% toluidine blue and 2% sodium borate in ddH20) at 60° C. for 3 min., mounted on slides and cover-slipped. Ultrathin sections were sectioned at 70 nm using a Leica Ultracut UCT ultramicrotome. Sections were mounted on 150 mesh copper grids, stained with uranyl acetate and lead citrate and viewed using a JEOL 1400 electron microscope. Images were captured using a Gatan digital camera. A blinded rater analyzed images by calculating the g-ratio (ratio of the diameter of the axon excluding the myelin sheath divided by the axon diameter including the myelin sheath) as well as counting the number of total axons, myelinated axons, dystrophic axons (defined as axon diameter>0.7 μm), degenerating axons and paranodes. Degenerating axons were identified as axonal swellings containing more than 5 clustered dark mitochondria and lysosomes. Paranodes were identified as axons with close proximity of the axolemma with the inner membrane of the myelin sheath with a surrounding cytoplasmic portion of oligodendrocyte.

### 1.1.8: MS Patient Cohort:

MS patients were recruited from the neuroimmunology clinical trial unit at the NeuroCure Clinical Research Center, Charite—Universitatsmedizin Berlin (Table 1). Inclusion criteria were MS based on the 2010 revised McDonald criteria, stable immunomodulatory therapy (RRMS) or no treatment (PPMS and SPMS). Exclusion criteria were acute relapse and/or corticosteroids within 6 months prior to inclusion. Disease course was determined under strict adherence to the 1996 Lublin criteria. Blood draws were fasting. The study was approved by the local ethics committee of Berlin (Landesamt für Gesundheit und Soziales (LAGeSo)). All study participants gave written informed consent. Studies were conducted in conformity with the 1964 Declaration of Helsinki in its currently applicable version.

TABLE 1
MS Cohort

| | | |
|---|---|---|
| MS patients | n | 180 |
| RRMS | n | 125 |
| PPMS | n | 23 |
| SPMS | n | 32 |
| Age (years) | Mean ± SD | 42.7 ± 9.4 |
| Sex (M/F) | n/n | 73/107 |
| Time since diagnosis (months) | Mean ± SD | 118.2 ± 87.6 |
| EDSS | Median (Min-Max) | 3.0 (0.0-8.0) |
| T2LC | Median (Min-Max) | 29 (0-162) |
| T2LV (ml) | Median (Min-Max) | 2790 (0-54,900) |
| CELC | Median (Min-Max) | 0 (0-6) |

Abbreviations:
MS, multiple sclerosis;
RRMS, relapsing-remitting MS;
PPMS, primary progressive MS;
SPMS, secondary progressive MS;
SD, standard deviation;
EDSS, Expanded Disability Status Scale;
T2LC, T2w lesion count;
T2LV, T2w lesion volume;
CELC, contrast enhancing lesion count

### 1.1.9 Magnetic Resonance Imaging (MRI):

MRI was performed at 1.5 Tesla using three-dimensional T1-weighted magnetization prepared for rapid acquisition and multiple gradient echo sequences (MPRAGE; T1w) and axial T2-weighted (T2w) sequences. Images were either acquired on a Sonata MRI (Siemens Medical Systems, Erlangen, Germany) with TE 4.38 ins, TR 2,110 ins, T1 1.1 ins, flip angle 150 and isotropic resolutions 1 mm³ for T1w, and Multiecho TSE with TE 8I ins, TR 5,780 ins, 1500 flip angle, resolution 0.5×0.5×3 mm, no gap for T2w, or on an Avanto MRI (Siemens Medical Systems, Erlangen, Germany) with TE 3.09 ins, TR 1,900 ins, T1 1.1 ins, flip angle 150 and isotropic resolutions 1 mm³ for T1w, and 3D TSE with TE 175 ins, TR 3,000 ins, flip angle 1200, isotropic resolutions 1 mm³ for T2w. Conventional spin-echo T1-weighted images (TR 1060 ins, TE 14 ins, 3 mm slice thickness, no gap and 44 contiguous axial slices) were obtained before and 5 minutes after injection of 0.1 mmol/kg Gd-DTPA (Magnevist, Bayer-Schering, Berlin, Germany).

T2w lesion segmentation was performed as using a semi-automated procedure including image co-registration using FLIRT (FMRIB Software Library, Oxford, UK) and inhomogeneity correction as embedded into the MedX v3.4.3 software package (Sensor Systems Inc., Sterling, VA, USA). Bulk white matter lesion load and lesion count of T2w scans were routinely measured using MedX.

For calculation of T1w/T2w ratio maps, MPRAGE, FLAIR and T2w scans were reoriented to standard space, bias field corrected and cropped to a robust field of view using FSL 5.0.9. The MPRAGE and FLAIR scans were then linearly co-registered to T2w using FSL FLIRT and then registered to MNI (Montreal Neurological Institute) space and brain extracted using the Brain Extraction Toolbox (BET). T2w lesions were then automatically segmented by applying the lesion prediction algorithm (LPA) to FLAIR scans, implemented in the Lesion Segmentation Toolbox version 2.0.15 for SPM. GM, WM and brain masks were then extracted from the MPRAGE. The lesion mask was subtracted from these masks to remove any lesion effects. The T1w/T2w ratio was created by dividing the processed MPRAGE scans by the processed T2w scans. Median T1w/T2w ratios were extracted from the normal appearing WM, GM and brain masks.

### 1.1.10: Targeted LC-MS/MS:

Serum samples for metabolomics analysis were prepared as described previously. Briefly, 50 μL serum (stored at −80° C.) and 200 μl ice cold extraction solvent (40% acetonitrile: 40% methanol: 20% H₂O), were vortexed for 2 minutes, then shaken in an Eppendorf shaker (Thermomixer R) at 1400 rpm, 4° C. for 1 hour and centrifuged at 4° C. for 10 minutes at ˜18,000×g in an Eppendorf microfuge. Supernatants were transferred to a clean tube and evaporated in a Speedvac (Acid-Resistant CentriVap Vacuum Concentrators, Labconco). Dried samples were stored at −80° C. Samples were resuspended in 100 μl of water containing the Internal Standards D7-Glucose at 0.2 mg/mL and H-Tyrosine at 0.02 mg/ml. Samples were resolved by LC-MS/MS, in negative mode at the optimum polarity in MRM mode on an electrospray ionization (ESI) triple-quadrupole mass spectrometer (AB Sciex 4000Qtrap, Toronto, ON, Canada). MultiQuant software (AB Sciex, Version 2.1) was used for peak analysis and manual peak confirmation. The results, expressed as area ratio (area of analyte/area of internal standard), were exported to Excel, and analyzed with MetaboAnalyst 3.0. Standard curves were prepared by adding increasing concentrations of GIcNAc or N-Acetyl-D-[UL-¹³C6]glucosamine ([UL¹³C₆] GIcNAc) (Omicron Biochemicals, Indiana) to 50 μl aliquot of control serum. This way we were able to create a calibration curve for HexNAc serum levels, obtaining absolute values rather than relative concentrations. Analysts were blinded in regard to sample origin (HC or MS).

### 1.1.11: Statistical Analysis

Statistical analyses for the in vitro and animal experiments were done with Graphpad Prism by t-tests, ANOVA with Sidak's post-test correction or comparing best fit curves from non-linear regression (Y=Bmax*X/(Kd+X) as described in the relevant figure legends. Statistical analyses for the clinical part were performed with R Project version 3.5.3. Correlations between serum HexNAc levels and lesion measurements were analyzed using nonparametric Spearman's Rho analysis. Correlations between HexNAc levels and T1w/T2w-ration measurements were analyzed using linear regression models with HexNAc levels as an independent variable.

### 1.2.1: GIcNAc and N-Glycan Branching Trigger Oligodendrogenesis by Inhibiting PDGFRα Endocytosis:

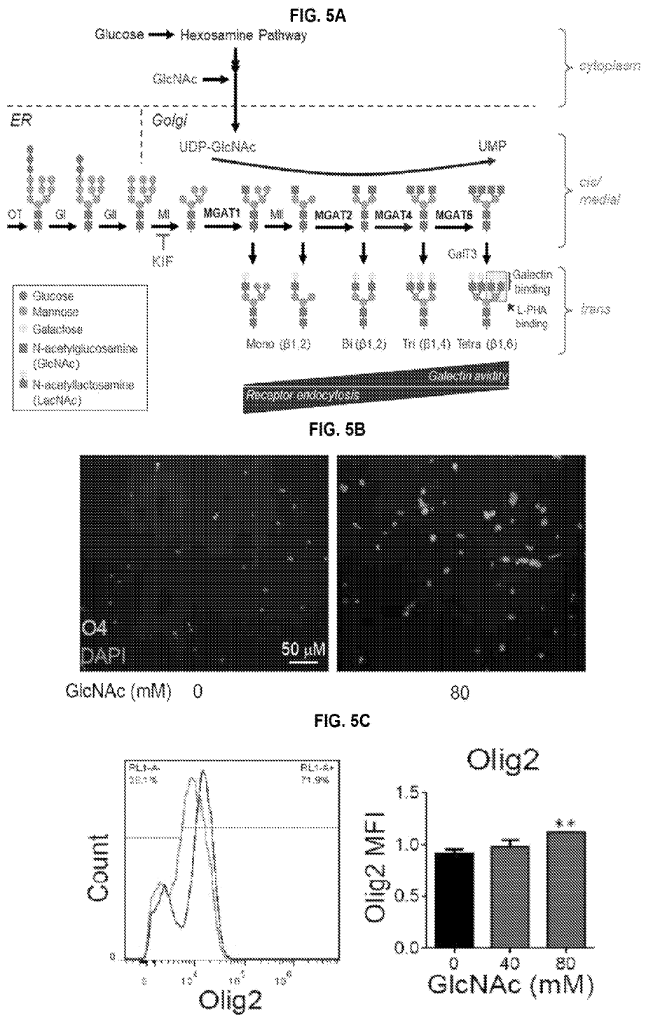

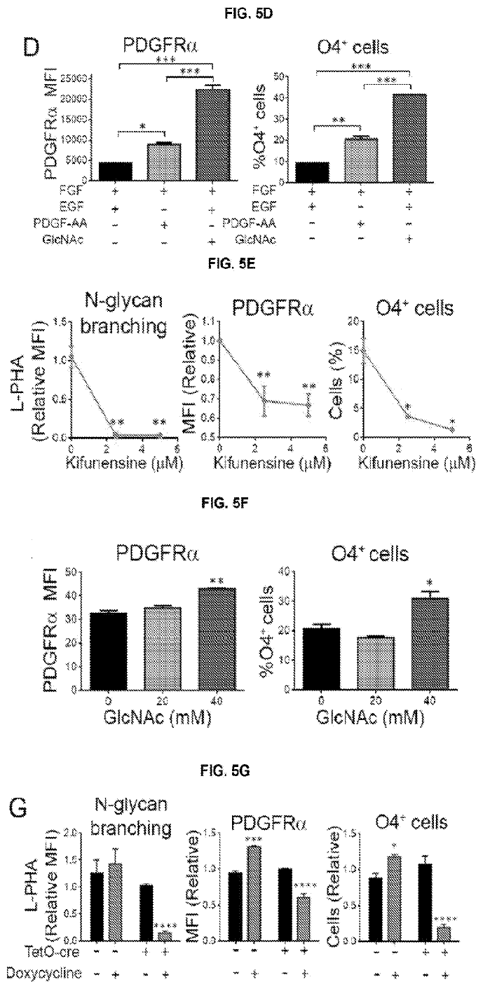

Oligodendrogenesis were examined in vitro using mouse neural stem cells (NSC) derived from the medial ganglionic eminence of E12.5 mouse embryos, where OPC's first appear. GIcNAc treatment of NSCs for 48 hrs in growth media lacking exogenous differentiation cytokines (i.e. no PDGF-AA, T3, CNTF) significantly increased N-glycan branching and PDGFRα surface expression (FIGS. 1A and 1B), the former assessed by L-PHA (Phaseolus vulgaris leukoagglutinin) flow cytometry (17,22). Consistent with increased PDGFRα surface expression, GIcNAc also promoted pre-oligodendrocyte differentiation as evidenced by augmented expression of Oligodendrocyte Transcription Factor (OLIG2) and increased numbers of 04 and GaIC positive cells (FIGS. 1A, 1B and 1C, FIGS. 5B and 5C). Double staining for PDGFRα and 04 revealed that GIcNAc promoted development of pre-oligodendrocytes (PDGFRαO4⁺) with no change in the number of OPC's (PDGFRα+O4⁻) (FIG. 1D). Thus, after only two days of culture, GIcNAc initiated oligodendrogenesis from NSC's despite the absence of exogenous differentiation cytokines such as PDGF-AA. Remarkably, GIcNAc in growth media was more potent than differentiation media containing exogenous PDGF-AA at initiating oligodendrogenesis (FIG. 5D). Combining GIcNAc with PDGF-AA also enhanced NSC differentiation to O4⁺ pre-oligodendrocytes (FIG. 5E).

To confirm a role for N-glycan branching in oligodendrogenesis, kifunensine was first used to inhibit N-glycan branching in NSC induced to differentiate by exogenous PDGF-AA. Reducing branching in NSC's using kifunensine significantly reduced PDGFRα surface expression and the number of O4′ cells induced by PDGF-AA differentiation media (FIG. 5F). To confirm this result genetically, Mgat5^{l} and doxycycline inducible Mgat1^{f/f}/tetO-cre/ROSA-rtTA mice were utilized. Mgat5 deletion mildly reduces N-glycan branching while Mgat1 deletion completely blocks N-glycan branching (FIG. 5A). In vitro doxycycline treatment of NSC from Mgat1^{f/f}/tetO-cre/ROSA-rtTA mice readily induced deletion of Mgat1, as measured by loss of L-PHA binding (FIG. 5G). Mgat5 and Mgat1 deleted NSC decreased surface levels of PDGFRα and were markedly reduced in their ability to differentiate into O4′ pre-oligodendrocytes in response to PDGF-AA differentiation media (FIG. 1E, FIG. 5G). In Mgat5 heterozygous NSC's, small reductions in N-glycan branching also inhibited oligodendrocyte differentiation (FIG. 1E). Thus, subtle changes in N-glycan branching can markedly impact oligodendrocyte differentiation from NSC in vitro.

### 1.2.2: GIcNAc and N-Glycan Branching Promote Primary Myelination in Mice

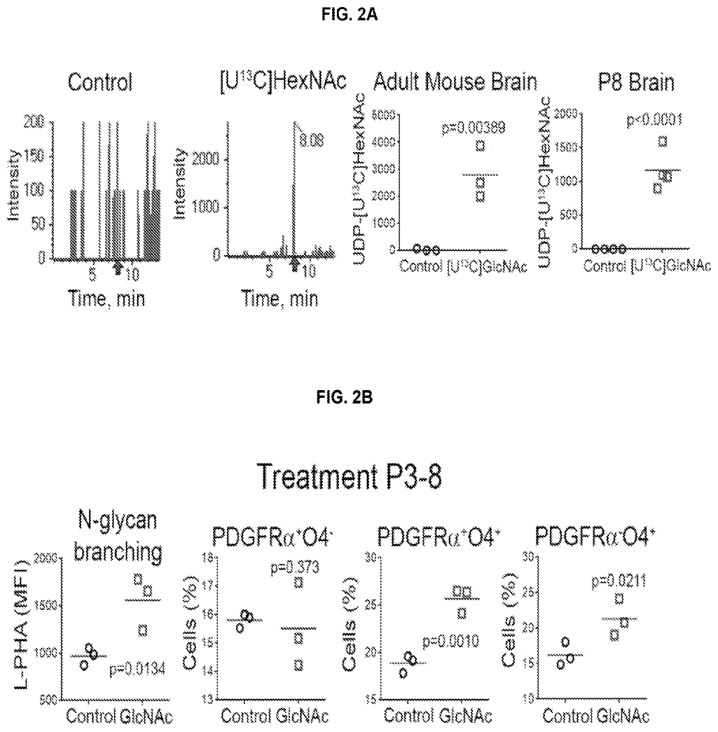

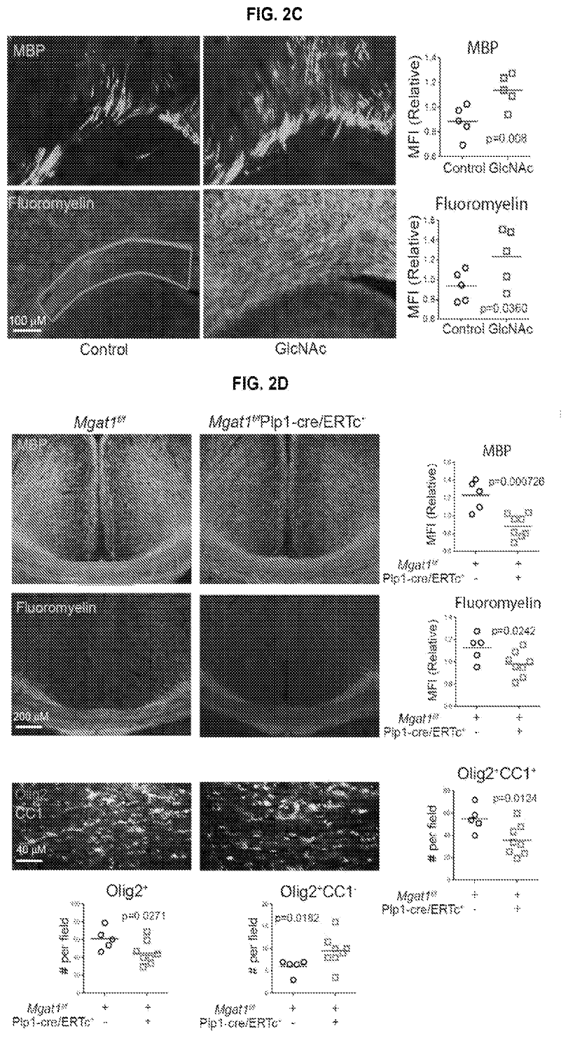

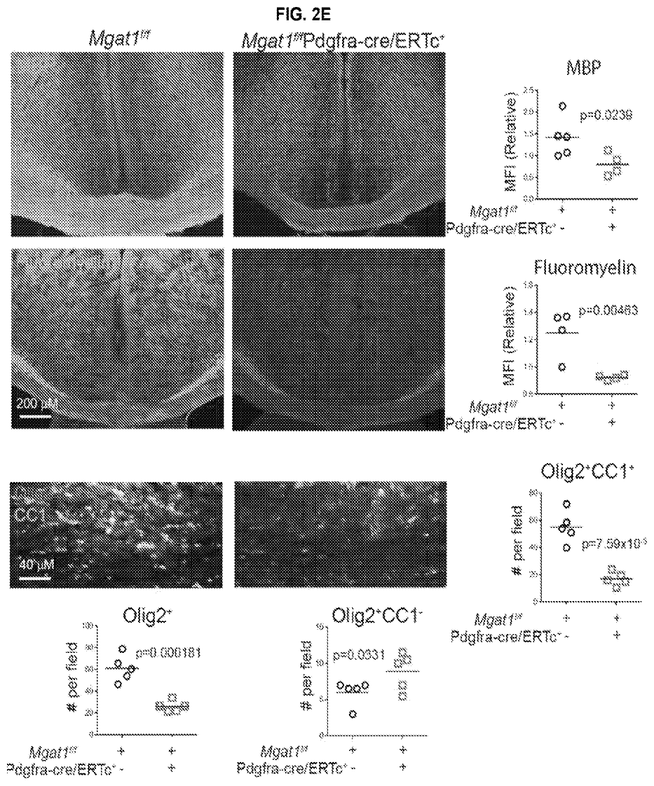

Next, whether oral GIcNAc can cross the blood-brain barrier to promote oligodendrocyte differentiation and myelination was examined in vivo. Adult mice (n=6) and lactating mothers were provided with/without C¹³-labelled GIcNAc ([U¹³C]GIcNAc) in their drinking water and metabolites derived from perfused brains were analyzed by Liquid chromatography—tandem mass spectroscopy (LC-MS/MS). Although this method does not resolve stereoisomers of N-Acetylhexosamines (ie. GIcNAc versus GaINAc), a reversible 4-epimerase (GALE) equilibrates UDP-GIcNAc and UDP-GaINAc in vivo. LC-MS/MS identified UDP-[U¹³C]-N-Acetylhexosamines (UDP-[U¹³C]-HexNAc) in treated adult female mouse brains as well as in the brains of their suckling pups (FIG. 2A). This demonstrates that orally delivered GIcNAc is not only able to cross the blood brain barrier and be metabolized to UDP-GIcNAc by CNS cells, it is also secreted at sufficient levels in breast milk to raise UDP-GIcNAc in the brains of suckling pups.

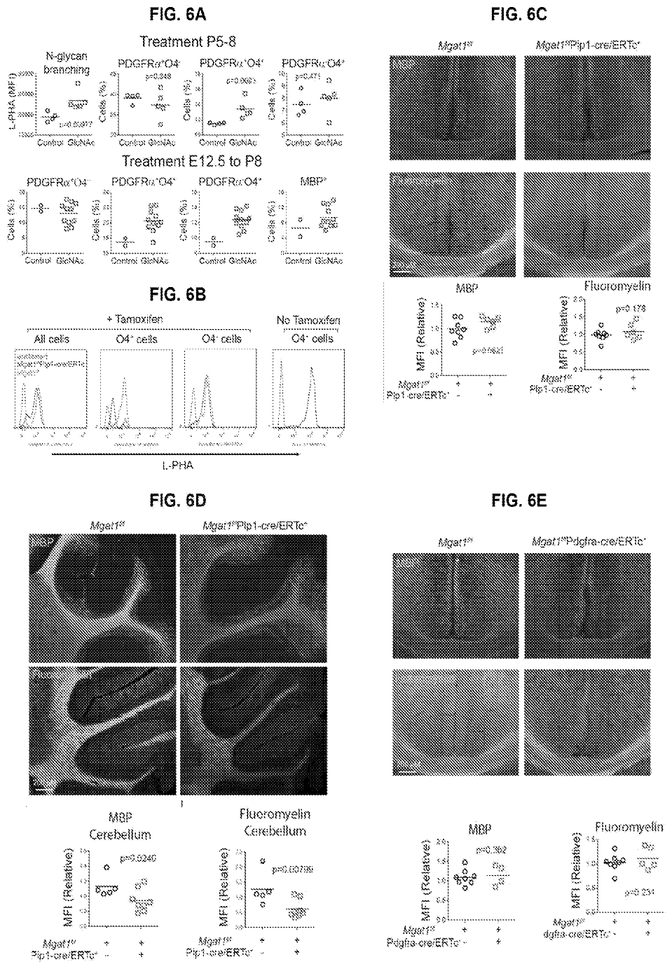

To assess whether oral GIcNAc promotes oligodendrogenesis in vivo in the absence of inflammation, primary myelination was examined in mice during the early perinatal period. GIcNAc or vehicle was provided to pregnant/lactating female mice from E12.5, postnatal day 3 (P3) or P5 through to P8. Indeed, oral GIcNAc increased N-glycan branching in PDGFRα⁺ cells as well as the number of pre-oligodendrocytes (PDGFRα⁺O4⁺), immature oligodendrocytes (PDGFRα-O4⁺) and mature oligodendrocytes (MBP*), with little effect on the number of OPC's (PDGFRα⁻O4⁺) (FIG. 2B and FIG. 6A). The lack of change in OPC number is consistent with our in vitro data (FIG. 1D) and suggests that GIcNAc promotes OPC self-renewal and/or NSC differentiation to OPC, resulting in a stable number of OPC's. Consistent with increased oligodendrogenesis, oral GIcNAc also increased primary myelination when provided to pups from P3-8, as assessed by increased levels of staining for myelin basic protein (MBP) and myelin (as measured by fluoromyelin) (FIG. 2C).

To confirm that N-glycan branching promotes myelination in the absence of inflammation in vivo, mice with tamoxifen inducible deletion of Mgat1 only in OPC's and oligodendrocytes, were generated, namely Mgat1^{f/f}° Plp1-cre/ERTc⁺ mice. As proteolipid protein (PLP) promoter driven Cre expression only becomes restricted to the oligodendrocyte lineage (OPC and oligodendrocyte) at P28, adult mice were focused on. OPC's continue to proliferate and generate significant new myelin in adulthood, with myelination gradually doubling from −2 to 10 months. Tamoxifen readily induced Mgat1 deletion in O4′ oligodendrocytes but not O4—cells in vivo, as determined by loss of L-PHA binding by flow cytometry (FIG. 6B). Consistent with slow accumulation of new myelin from OPC's during adulthood, two weeks following tamoxifen treatment (Mgat1 deletion) adult Mgat1^{f/f}Plp1-cre/ERTc⁺ mice did not display significant differences in brain levels of MBP or myelin (fluoromyelin) relative to tamoxifen treated controls (FIG. 6). However, eight weeks after initial tamoxifen treatment, Mgat1 deletion resulted in significant reductions in levels of MBP, myelin (fluoromyelin) and number of total (Olig2⁺) and mature (Olig2⁺CC1⁺) oligodendrocytes along with increased numbers of immature (Olig2⁺CC1⁻) oligodendrocytes (FIG. 2D, FIG. 6D). To confirm that these results primarily arose from a defect in new myelin formation from OPC's, rather than a defect in mature oligodendrocytes, we generated Mgat1^{f/f}Pdgfra-creER⁺ mice where tamoxifen induces deletion of Mgat1 in OPC's but not mature oligodendrocytes. Indeed, eight weeks but not two weeks after tamoxifen treatment, deletion of Mgat1 in OPC's significantly reduced levels of MBP, myelin (fluoromyelin) and number of total (Olig2⁺) and mature (Olig2⁺CC1⁺) oligodendrocytes along with increased numbers of immature (Olig2⁺CC1⁻) oligodendrocytes (FIG. 2E, FIG. 6E). Tamoxifen has been reported to promote myelination(47), however Mgat1 deletion reduced myelination despite potential positive effects of tamoxifen. Together, these data demonstrate that GIcNAc and N-glycan branching promote primary myelination in mice by driving OPC differentiation.

### 1.2.3: GIcNAc Prevents Damage to Demyelinated Axons by Promoting Myelin Repair

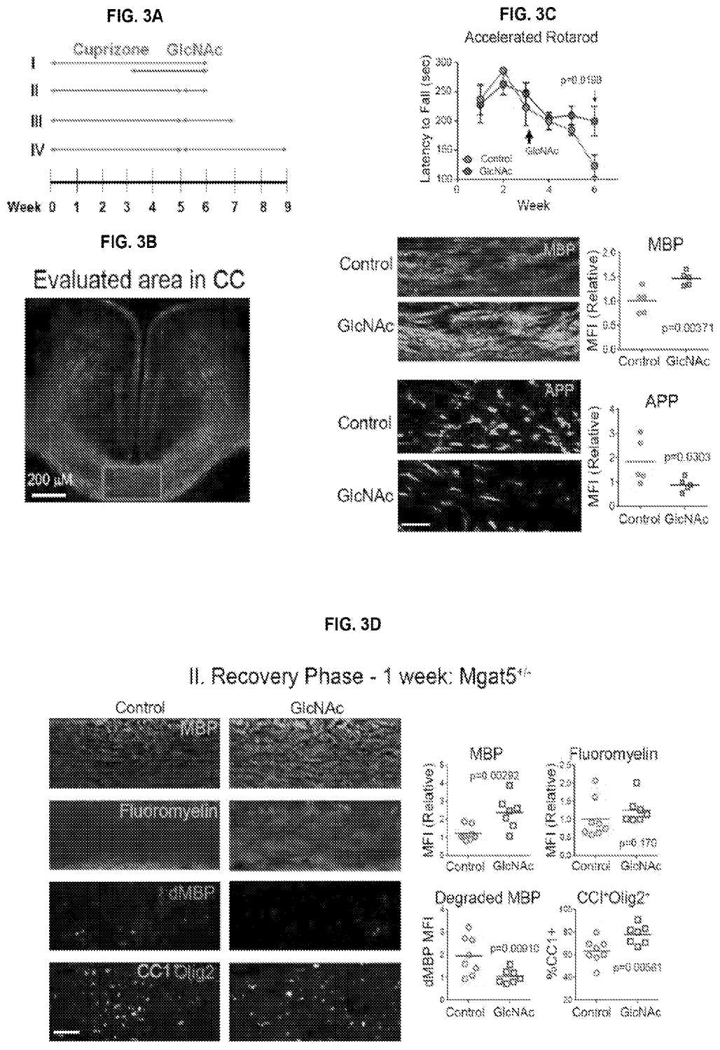

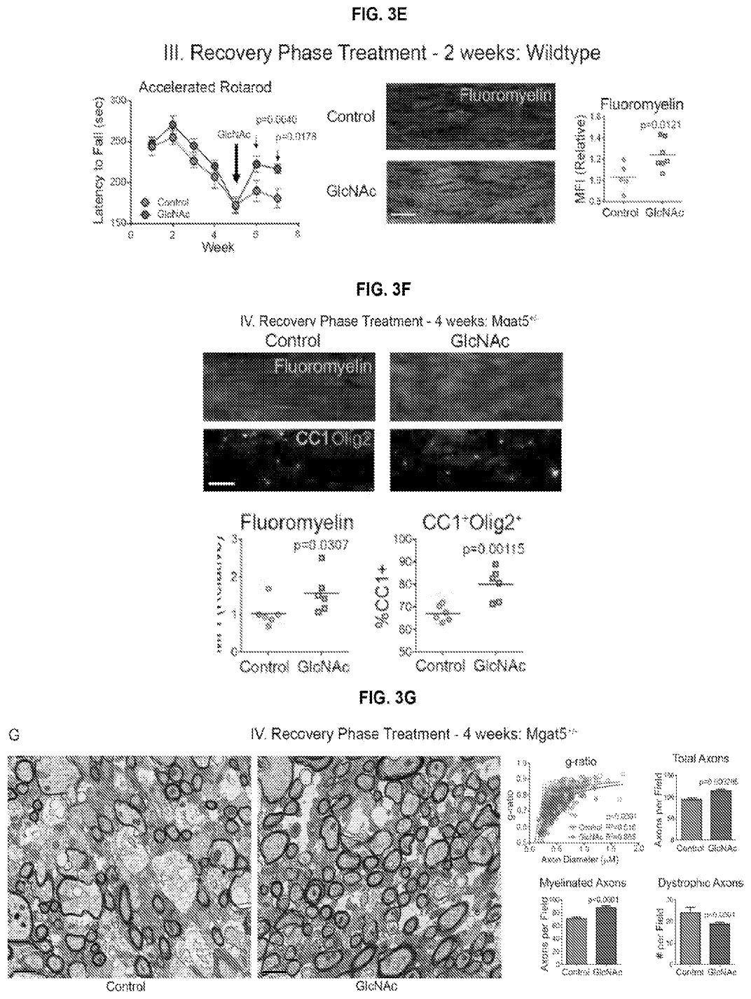

To explore whether GIcNAc can promote remyelination in adult mice following myelin injury, the cuprizone model of non-immune induced de-myelination/re-myelination was utilized on Mgat5⁺/−and wildtype C57BL/6 mice. Cuprizone at 0.2% induces demyelination in the corpus callosum by 3 weeks, with maximum demyelination at 5-6 weeks. Partial re-myelination via maturation of OPC's begins at the height of demyelination and becomes complete ˜3-5 weeks after cuprizone withdrawal. Given this, four different treatment regimens we examined (FIG. 3A). When GIcNAc was concurrently provided during the final 3 weeks of a 6-week cuprizone (0.2%) exposure in wildtype mice, GIcNAc prevented loss of motor function (as measured using rotarod fall latency) while increasing MBP levels and reducing axonal damage (as measured by reduced accumulation of amyloid precursor protein (APP)) in the corpus callosum (FIG. 3C). To address potential confounding effects of GIcNAc on inhibiting demyelination by cuprizone during concurrent treatment, treatment of wildtype mice were initiated for 2 weeks or Mgat5^{+/-} mice for 1 or 4 weeks of GIcNAc only after cuprizone was stopped (FIG. 3A). This revealed that GIcNAc enhanced levels of MBP, myelin (fluoromyelin) and mature oligodendrocytes (CC1*Olig2⁺) while reducing the amount of degraded MBP (dMBP)/myelin degeneration within the corpus callosum (FIGS. 3D, 3E, and 3F). dMBP was detected by an antibody that specifically recognizes areas of myelin degeneration.

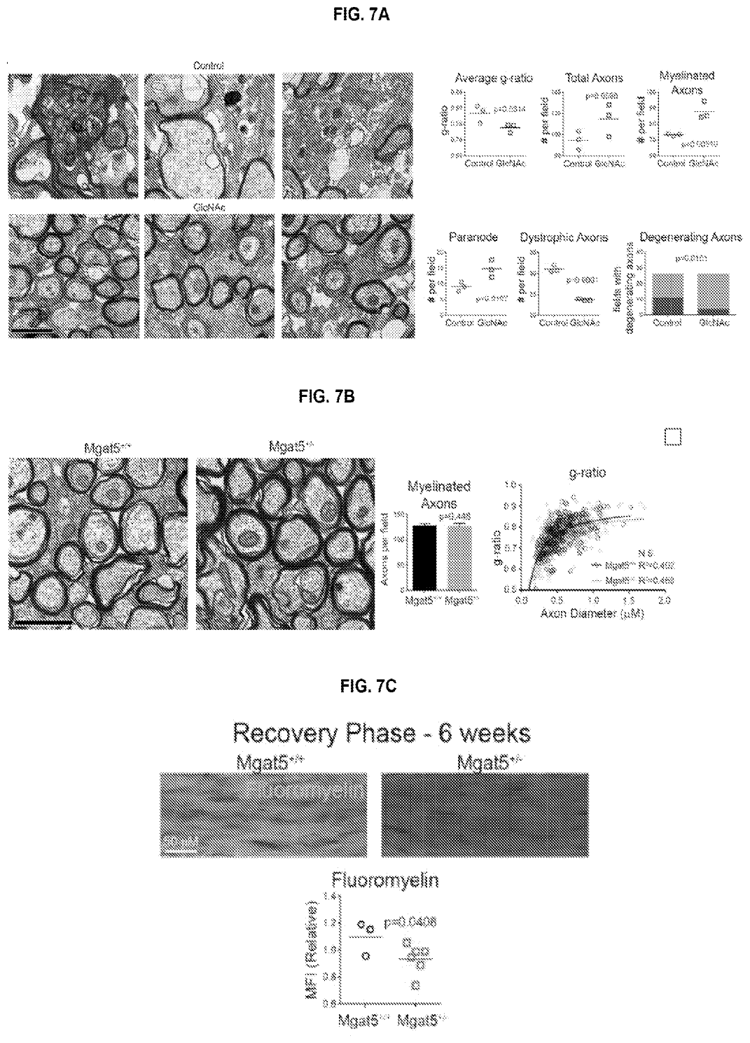

Electron microscopy analysis confirmed these results, revealing that GIcNAc enhanced the number of myelinated axons, and the degree of myelination as measured by the g-ratio, while also reducing axon loss and the number of degenerating and dystrophic/swollen axons (FIG. 3G, FIG. 7A). GIcNAc also enhanced the number of paranodes, which increase with re-myelination (FIG. 7A). Enhancement of myelination by GIcNAc depends on time, as the increase in fluoromyelin staining in Mgat5^{+/-} mice was ˜2-fold greater with 4 versus 1 week of GIcNAc treatment (FIGS. 3D and 3F). Importantly, the subtle reductions in N-glycan branching induced in Mgat5^{+/-} mice did not alter baseline levels of myelin yet reduced re-myelination following cuprizone induced injury relative to Mgat5^{+/+} control mice (FIGS. 7B and 7C). Together, these data indicate that GIcNAc and N-glycan branching promotes myelin repair and provides neuro-protection to axons following demyelination.

1.2.4: A Marker of Serum GIcNAc Inversely Associates with Imaging Markers of Myelin-Axon Damage.

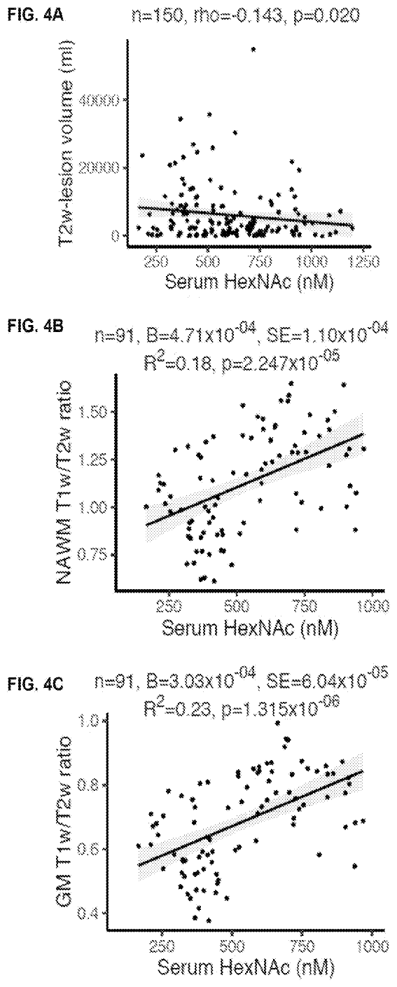

To explore whether alterations in GIcNAc may impact myelination status in MS patients, a cohort of 180 MS patients was used to correlate endogenous serum HexNAc levels with measures of white matter damage by magnetic resonance imaging (MRI) of the brain. Increased T2w lesion volume and count on brain MRI are measures of the extent and frequency of demyelination, respectively. T2w lesion volume correlated with lower HexNAc serum levels (FIG. 4A, p=0.020), whereas T2w lesion count did not (p=0.387). Likewise, patients with contrast enhancing lesions, a marker of active inflammation in MS, had similar serum HexNAc levels to those without (p=0.866), suggesting GIcNAc primarily impacts the extent of permanent demyelination rather than initiation of inflammatory demyelination. T1w/T2w ratio maps reflect microstructural integrity of myelin/axons in normal appearing white matter (NAWM) and cortical grey matter. With age and gender as covariates, low serum HexNAc levels were strongly associated with lower T1w/T2w ratios indicating microstructural damage of myelin/axons in both normal appearing white matter (r2=0.18, p=20.25×10−5) and grey matter (r2=0.23, p=1.3²×10−6), (FIGS. 4B and 4C). Together, these data are consistent with our mouse data and suggest that GIcNAc may promote myelination in MS.

2.3: Described herein the present invention features a novel pathway for regulating oligodendrogenesis, primary myelination and myelin repair by N-glycan branching and GIcNAc. The present invention demonstrates that GIcNAc and N-glycan branching are neuroprotective for demyelinated axons by promoting oligodendrogenesis and myelination from OPC's. The association of low endogenous GIcNAc with increased myelin-axon microstructural damage in MS patients suggests this mechanism is relevant to pathogenesis of MS. Furthermore, low levels of serum GIcNAc in MS patients is associated with a progressive disease course, clinical disability and multiple neuroimaging measures of neurodegeneration.

### 2.1: Open-Label Phase 1 Dose Finding Trial of Oral GIcNAc in Multiple Sclerosis.

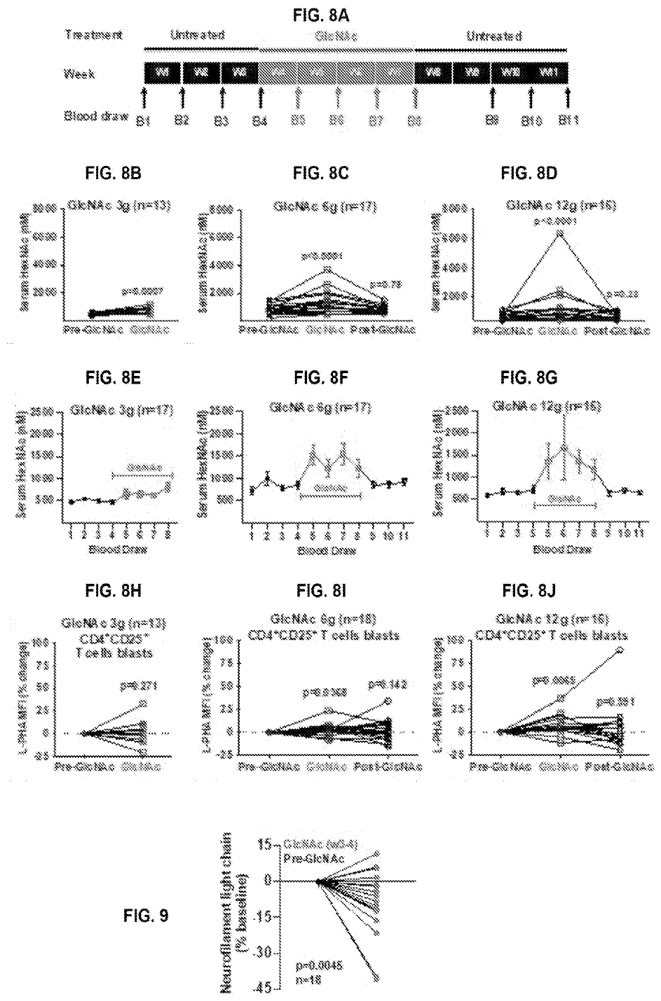

Based on its immunomodulatory effects, a FDA approved (IND 122235) dose escalation was carried out, open-label NIH funded Phase 1 mechanistic clinical trial of oral GIcNAc in MS patients (RRMS and PMS) on glatiramer acetate and not in acute relapse, with the primary endpoint an increase in N-glycan branching in T cells. This endpoint was used as a surrogate marker for GIcNAc's biological activity. Three doses were evaluated: 1 g TID (3 g total, n=13), 2 g TID (6 g total, n=18) and 4 g TID (12 g total, n=16). Subjects had baseline blood drawn weekly four times, then started on oral GIcNAc with 4 weekly blood draws (FIG. 8A). The 6 g and 12 g cohorts also had a four-week washout period, with three additional blood draws (FIG. 8A).

### 2.2: Oral GIcNAc was Safe and Well Tolerated by MS Patients.

Compliance was near 100% and oral GIcNAc was found to be safe at all doses, with only mild GI symptoms (gas, bloating, loose stool) in ˜50% of subjects in the 12 g group but not the other doses. No subject discontinued oral GIcNAc due the GI symptoms. The annualized relapse rate on GIcNAc in the combined group was 0.277 (from a single relapse in a subject on the 6 g dose).

### 2.3: Oral GIcNAc Raises Serum Levels and N-Glycan Branching in Blasting CD4⁺ T Cells.

Compared to baseline, all three doses of oral GIcNAc readily increased serum HexNAc levels with effect size increasing in a dose-dependent manner from 37% to 65% to 112% (relative to baseline) for the 3 g, 6 g and 12 g cohorts respectively (FIGS. 8B, 8C, 8D, 8E, 8F, and 8G). Serum HexNAc levels rapidly declined back to baseline during the washout period (FIGS. 8B, 8C, 8D, 8E, 8F, and 8G), demonstrating that elevated serum GIcNAc levels require regular intake of oral GIcNAc.

To assess the primary endpoint (increased N-glycan branching in T cells), flow cytometry was used to assess binding of the plant lectin L-PHA (Phaseolus vulgaris leukoagglutinin) to T cells. L-PHA is a highly sensitive and specific measure of N-glycan branching. L-PHA binding was increased in blasting/activated CD25⁺CD4⁺ T cells in the 12 g (p=0.0065) and 6 g (p=0.037) but not the 3 g cohort, with average increases of 7%, 3% and 1.7% respectively (FIGS. 8H and 8I); the latter indicative of a dose response. Note that in mice, oligodendrocyte precursor cells are significantly more sensitive to oral GIcNAc than T-cells, suggesting that the observed changes in T-cells likely significantly underestimate the effects of oral GIcNAc on raising N-glycan branching in OPC's in humans. In contrast to blasting/activated CD25⁺CD4⁺ T cells, activated CD25⁺CD8⁺ T cells, resting CD25-CD4⁺ and CD25-CD8⁺ T cells and activated/resting CD19⁺ B cells displayed no increase in L-PHA binding (data not shown). This result directly parallels our pre-clinical data in mice, whereby within the immune system, oral GIcNAc primarily increases N-glycan branching in blasting/activated T-cells, while having minimal or no effects on resting CD4⁺ T cells, CD8⁺ T cells and B cells. This provides specific targeting of activated CD4⁺ T cells within the immune system and is secondary to two factors: 1) cellular uptake of GIcNAc by macropinocytosis depends on the rate of membrane turnover, which is high in blasting/activated T cells but not resting lymphocytes and 2) baseline N-glycan branching is significantly lower in CD4⁺ T cells than CD8⁺ T cells and CD19′ B cells, making them more susceptible to GIcNAc availability.

During the washout period (post-GIcNAc), L-PHA binding to blasting/activated CD25⁺CD4⁺ T cells showed mixed directional changes with no significant difference when compared to either pre-GIcNAc or during GIcNAc treatment (FIGS. 8H and 8I). Note that alterations in N-glycan branching at the cell surface is delayed relative to changes in GIcNAc availability as it requires subsequent changes in metabolic production of UDP-GIcNAc, new glycoprotein production and membrane turnover.

### 2.4: Exploratory Retrospective Analysis: Neurofilament Light Chain.

Given the recent pre-clinical and observational human data suggesting that GIcNAc may promote myelination and inhibit neurodegeneration, therefore, serum from all cohorts was retrospectively assessed for levels of neurofilament light chain (Nfl). Neurons exclusively express Nfl and release it when damaged, with high serum Nfl levels (>˜40 pg/ml) serving as a robust biomarker for ongoing neuro-axonal damage in MS. This analysis was done blinded by Jens Kuhle (University of Basel), who has published extensively on Nfl in MS. In subjects with elevated baseline Nfl levels from all three dose groups, indicative of active ongoing neuro-axonal damage, oral GIcNAc significantly reduced serum Nfl levels during week 3 and 4 of treatment (p=0.0045, n=18, FIG. 9). Effects of GIcNAc at week 3-4 of oral GIcANc was chosen for assessment based on our preclinical data, which revealed that stimulation of myelin repair and associated neuroprotection in mice was greatest following 3-4 weeks of oral GIcANc treatment.

### 2.5: Exploratory Retrospective Analysis: Confirmed Improvement in Clinical Disability.

Four-week confirmed improvement in disability (EDSS) was also evaluated in the 6 g and 12 g cohorts. The 3 g cohort did not have a washout period and therefore confirmed changes in EDSS could not be evaluated. Note that no current FDA approved MS therapeutic has been shown to improve disability. Baseline EDSS scores were compared with scores after 4 weeks of oral GIcNAc that were confirmed 4 weeks later at the last study visit. Interestingly, 4 of 18 (22%) subjects at the 6 g/day and 5 of 16 (31%) subjects at the 12 g/day dose had a 4-week confirmed improvement in their clinical disability (EDSS). Although this data is unblinded data and therefore biased, improvement in disability would be consistent with an effect of GIcNAc on re-myelination.

The present invention features clinical data that suggests that oral GIcNAc may promote myelin repair and neuroprotection in MS but dose is limited at the 12 g dose by microbiota metabolizing GIcNAc (leading to patient bloating, gas, loose stool).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of treating a demyelinating disease or repairing myelin in a subject in need of such treatment, the method comprising administering to the subject a therapeutic amount of a composition comprising N-acetylglucosamine (GlcNAc), GlcNAc-6-acetate, or a combination thereof.

2. The method of claim 1, wherein the subject is human.

3. The method of claim 1, wherein the demyelinating disease is multiple sclerosis (MS), optic neuritis, myelin oligodendrocyte glycoprotein antibody associated disease, neuromyelitis optica spectrum disorder, transverse myelitis, or acute disseminated encephalomyelitis.

4. The method of claim 1, wherein the composition is administered intravenously.

5. The method of claim 4, wherein the composition is administered at a dose of about 1 g to about 150 g.

6. The method of claim 1, wherein the composition is administered orally.

7. The method of claim 6, wherein the composition is administered at a dose of about 1 g to about 20 g.

8. The method of claim 1, wherein the composition is administered once daily, twice daily, or three times daily.

9-13. (canceled)

14. A method of improving brain development and/or cognitive function in an infant, child, or adult, the method comprising orally administering to the infant, child or adult a therapeutic amount of N-acetylglucosamine (GlcNAc).

15. The method of claim 14, wherein GlcNAc is incorporated into baby formula.

16. The method of claim 14, wherein GlcNAc improves myelination in the infant's brain.

17. The method of claim 14, wherein GlcNAc improves cognitive function in the infant, child or adult.

18. (canceled)

19. A composition comprising N-acetylglucosamine (GlcNAc), GlcNAc-6-acetate, or a combination thereof.

20. A method of promoting N-glycan branching in neural stem cells, oligodendrocyte precursor cells, oligodendrocytes, or a combination thereof in a subject in need thereof, the method comprising administering to the subject a therapeutic amount of the composition according to claim 19.

21-24. (canceled)

25. The method of claim 1, wherein the composition promotes N-glycan branches in neural stem cells, oligodendrocyte precursor cells, oligodendrocytes, or a combination thereof.

26-32. (canceled)
